(19) United States
(12) Patent Application Publication
(10) Pub. No.: US 2002/0086040 A1
(43) Pub. Date: Jul. 4, 2002

(54) Cosmetic composition which provides good hold properties and which comprises a copolymer with an acid unit

(76) Inventors: Christine Dupuis, Paris (FR); Henri Samain, Bievres (FR); Bertrand Lion, Luzarches (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/984,552

(22) Filed: Oct. 30, 2001

(30) Foreign Application Priority Data
Oct. 30, 2000 (FR) 00 13937

(51) Int. Cl. A61K 7/06; A61K 7/00

(52) U.S. Cl. 424/401

(57) ABSTRACT

A cosmetic composition comprising, in a cosmetically acceptable medium, at least one film-forming polymer (A) chosen so that a material, obtained by drying the at least one polymer (A) in an aqueous or alcoholic medium at ambient temperature and at a relative humidity of 50%±5%, exhibits a mechanical profile defined by at least:

(a) an elongation at break (ε_{b}) of greater than or equal to 160%;

(b) an instantaneous recovery (Rᵢ) of from 25 and to 70% after an elongation of 150%; and

(c) a recovery (R₃₀₀) at 300 seconds of from 30 and to 100% after an elongation of 150%;

wherein the at least one film-forming polymer (A) comprises at least one monomeric residue comprising an unsaturated acid unit, the acid number of which is greater than 82. Processes for obtaining such cosmetic compositions are also disclosed.

The subject matter of this invention is a cosmetic composition comprising, in a cosmetically acceptable medium, at least one film-forming polymer (A). It is also targeted at a process for shaping or retaining the form of the hair using this composition and at its use in the formulation of styling products, such as lacquers, sprays or mousses, for the purpose of obtaining retention of the form or shaping of the hairstyle.

Mention may be made, among the hair products for fixing the hair which are the most widely available in the cosmetics market, of compositions to be sprayed as an aerosol or as a pump-action spray, such as lacquers, sprays or mousses, composed essentially of a solution, generally an alcoholic or aqueous/alcoholic solution, of a film-forming polymer which is soluble in water or in the alcohol, as a mixture with various cosmetic adjuvants.

However, these hair formulations, such as mousses, gels and, in particular, aerosol sprays and lacquers, intended to retain the form of the hairstyle, still may not allow the hairstyle to withstand in a completely satisfactory manner the various natural movements of everyday life, such as walking, head movements or gusts of wind.

The polymers used for the formulation of these hair products are anionic, amphoteric or nonionic film-forming polymers which may result in the formation of films having a more or less hard and brittle nature.

When the polymer is too brittle, the percentage of elongation at break measured on the film is low, i.e., generally less than 2%, and the hold of the hairstyle over time is not assured.

To overcome this problem, these polymers have been used in combination with plasticizers and as a result more flexible, less brittle coatings have been obtained. However, these films may be deformable and plastic, such that, after deformation, they only recover their initial form to a very slight extent. In such cases, while the hold of the hairstyle is improved, it is still not completely satisfactory since the form of the hairstyle changes over time.

More satisfactory results in terms of hold have been obtained with compositions comprising a combination of film-forming polymers, such as, for example, a polyvinylcaprolactam polymer and an acrylic polymer. However, these compositions may still not be entirely satisfactory, in so far as they may cause the hair to lose some of its natural cosmetic properties. In addition, it would be desirable to further increase the fixing power.

Furthermore, the polymers used to date in styling compositions can exhibit the disadvantage of being poorly removed even after shampooing.

There is, therefore, a desire to find cosmetic compositions for the retention of the form of and/or the fixing of the hairstyle which provide the hair with, in addition to durable fixing, at least one good cosmetic property, in particular good disentangling, softness and a pleasant and natural appearance, and which give a better performance than those of the prior art with regard to the criterion of removal on shampooing.

Surprisingly and unexpectedly, the inventors have discovered that it is possible to overcome at least one of the technical problems mentioned above by using the polymers disclosed herein.

One aspect of this invention is a cosmetic composition comprising, in a cosmetically acceptable medium, at least one film-forming polymer (A) chosen so that a material, obtained by drying the at least one polymer (A) in an aqueous or alcoholic medium at ambient temperature and at a relative humidity of 50%±5%, exhibits a mechanical profile defined by at least:

(a) an elongation at break (ε_{b}) of greater than or equal to 160%;

(b) an instantaneous recovery (Rᵢ) of from 25 to 70% after an elongation of 150%; and

(c) a recovery (R₃₀₀) at 300 seconds of from 30 to 100% after an elongation of 150%.

The at least one film-forming polymer (A) comprises at least one monomeric residue comprising an unsaturated acid unit, the acid number of which is greater than 82.

Another aspect of the present invention relates to a process for shaping or retaining the form of a hairstyle comprising the use of this composition.

Yet another aspect of the present invention relates to the use of this composition in the manufacture of cosmetic compositions, in particular hair compositions intended for the retention of the form of or for shaping a hairstyle.

Yet another aspect of the present invention relates to a process for the manufacture of film-forming polymers and the cosmetic compositions comprising them.

Within the meaning of the present invention, the term “film obtained by drying at ambient temperature and at a relative humidity of 50%±5%” is understood to mean the film obtained under these conditions starting from a mixture comprising 6% of active material (a.m.) of at least one film-forming polymer (A) in an aqueous, i.e., water, medium or an alcoholic medium, such as ethanol, the amount of mixture being adjusted in order to obtain, in a teflon matrix, a film with a thickness of 500±50 μm. The drying is continued until the weight of the film no longer changes, which represents approximately 12 days. The at least one polymer (A) which is soluble or partially soluble in an alcoholic medium, such as ethanol, is tested in the alcoholic medium. The other polymers are tested in water, in a soluble or dispersed form. The term “ambient temperature” is defined as 22±2° C.

Within the meaning of the present invention, the degree of elongation at break and the degree of recovery are evaluated by means of the tests described below.

To carry out the tensile tests, the film is cut up into test specimens of rectangular shape, with a length of 80 mm and a width of 15 mm.

The tests are carried out on a device sold under the name Lloyd, reference LR5K, or sold under the name Zwick, under the same temperature and humidity conditions as for the drying, that is to say a temperature of 22±2° C. and a relative humidity of 50±5%.

The test specimens are stretched at the rate of 20 mm/min and the distance between the clamping jaws is 50±1 mm.

The procedure for determining the instantaneous recovery (Rᵢ) is as follows:

the test specimen is stretched by 150% (max), i.e., 1.5 times its initial length (l₀),

the stress is released while imposing a return rate equal to the pull rate, i.e., 20 mm/min, and the elongation of the test specimen is measured as a percentage, after returning to zero load (εᵢ).

The instantaneous recovery in % (Rᵢ) is given by the formula below:

Rᵢ=((εₘₐₓ−εᵢ)/εₘₐₓ)×100

To determine the recovery at 300 seconds, the test specimen which has been subjected to the preceding operations is maintained at zero stress for an additional 300 seconds and its degree of elongation is measured as a percentage (ε₃₀₀).

The recovery at 300 seconds in % (R₃₀₀) is given by the formula below:

R₃₀₀=((εₘₐₓ−ε₃₀₀)/εₘₐₓ)×100

In the compositions in accordance with the invention, the at least one film-forming polymer (A) is, for example, present at a concentration ranging from 0.05 to 20% by weight with respect to the total weight of the composition. In other embodiments, the concentration of the at least one film-forming polymer (A) ranges from 0.1 to 15% by weight or from 0.25 to 10% by weight with respect to the total weight of the composition.

The at least one film-forming polymer (A) comprises at least one monomeric residue which is, for example, chosen from monomeric residues comprising at least one unsaturated carboxylic acid and monomeric residues comprising at least one unsaturated sulphonic acid.

The at least one film-forming polymer (A) comprises at least one monomeric residue comprising at least one unsaturated acid unit, for example, a carboxylic acid unit, and further, for example, an acrylic acid unit and an methacrylic acid unit.

The acid number of the at least one unsaturated acid ranges, for example, from 82 to 235 and further, for example, from 90 to 190 and even further, for example, from 100 to 130.

The degree of elongation at break (ε_{b}) ranges, for example, from 160 to 10,000%.

The at least one film-forming polymer (A) is, for example, soluble in an aqueous or aqueous/alcoholic medium. The at least one film-forming polymer(A) may be completely or partially neutralized.

Another aspect of the invention relates to a process for the manufacture of at least one film-forming polymer by carrying out the following polymerizations in an alcoholic or aqueous/alcoholic solvent medium in the presence of a free radical initiator at a temperature ranging from 60° C. to 90° C.:

first polymerizing at least one monomer (i) comprising at least one hydrophilic unit to obtain a polymer rich in hydrophilic units;

subsequently polymerizing the polymer rich in hydrophilic units with a mixture of at least one hard monomer (ii) and at least one soft monomer (iii) to obtain said at least one film-forming polymer; said monomers (i), (ii) and (iii) being used in the following proportions, defined by weight with respect to the total weight of the monomeric residues in the at least one film-forming polymer:

10 to 50% of said at least one monomer (i) comprising at least one hydrophilic unit;

20 to 50% of said at least one hard monomer (ii); and

30 to 70% of said at least one soft monomer (iii).

Within the meaning of the present invention, the term “monomer comprising at least one hydrophilic unit” is understood to mean a monomer chosen from monomers having 3 to 8 carbon atoms and comprising at least one unit chosen from a monocarboxylic unit and a dicarboxylic acid unit. Such monomers may be acrylic acid, methacrylic acid or itaconic acid. In one embodiment of the invention, the monomer is acrylic acid.

The term “hard monomer” is understood to mean methacrylates chosen from linear C₁ to C₃ alkyl methacrylates, cyclic C₆ to C₈ alkyl methacrylates, and branched alkyl methacrylates chosen from the methacrylates forming, by polymerization as described in the work “Chimie Macromoleculaire [Macromolecular Chemistry],” G. Champetier, published by Hermann, a homopolymer with a glass transition temperature of greater than 50° C. For example, a hard monomer, as defined herein, is methyl methacrylate.

The term “soft monomer” is understood to mean acrylates chosen from linear C₁ to C₁₂ alkyl acrylates, cyclic C₆ to C₇ alkyl acrylates, and branched alkyl acrylates chosen from the said acrylates forming, by polymerization as described in the work “Chimie Macromoleculaire [Macromolecular Chemistry],” G. Champetier, published by Hermann, a homopolymer with a glass transition temperature of less than 25° C. Examples of a soft monomer, as defined herein, include methyl acrylate and isobutyl acrylate.

The monomers (i), (ii) and (iii) are, for example, used in the following proportions, defined by weight with respect to the total weight of the monomers:

15 to 30% of at least one monomer (i) comprising at least one hydrophilic unit;

25 to 40% of at least one hard monomer (ii); and

35 to 55% of at least one soft monomer (iii).

The at least one film-forming polymer obtained by polymerization of the monomers (i), (ii) and (iii) can have, for example, a weight average molecular mass, ranging for example, from 40,000 to 150,000, and as a further example, from 80,000 to 110,000.

Use can be made, as free radical initiator, of 2,5-bis(2-ethylhexanoylperoxy)-2,5-dimethylhexane.

The proportion of the free radical initiator can range, for example, from 0.6 to 2% by weight with respect to the weight of the monomers, and as a further example, about 1.1%.

The solvent used in carrying out the process according to the invention can be, for example, ethanol.

The proportion of solvent can, for example, range from 40 to 60% with respect to the total weight of the monomers.

According to a representative form of the process in accordance with the invention, the polymerization can be, for example, carried out at 78° C. under refluxing ethanol, the concentration of which can be, for example, ranging from 30 to 60% and as a further example, about 50%, such as by the following protocol:

the acrylic acid is introduced into a reactor, the introduction time ranging from 20 to 45 minutes, such as about 30 minutes, with 18 to 20% of the total amount of the initiator;

polymerization is allowed to take place at 78° C. for 15 to 45 minutes, such as for 30 minutes;

a mixture of hard and soft monomers is subsequently introduced into the reactor, the introduction time ranging from 1 hour to 2 hours, such as about 1 hour 30 minutes, with 80 to 82% of the total amount of the initiator;

polymerization is allowed to take place for an additional 3 to 4 hours to provide for complete consumption of the monomers,

the reaction medium is diluted with ethanol to obtain at least one film-forming polymer at a concentration of 20%.

Another aspect of the invention is at least one film-forming polymer capable of being obtained in accordance with the process according to the invention in the preparation of at least one film-forming polymer.

In the compositions in accordance with the invention, the cosmetically acceptable medium comprises, for example, water or at least one cosmetically acceptable solvent, such as alcohols, or mixtures of water and at least one solvent. Representative solvents include, for example, C₁-C₄ alcohols.

Mention may be made, among these alcohols, of ethanol and isopropanol. In one embodiment of the invention, ethanol or a mixture of water and ethanol is used.

The composition of the invention can also comprise at least one additive chosen from thickeners, anionic, nonionic, cationic and amphoteric surfactants, fragrances, preservatives, sunscreen agents, proteins, vitamins, provitamins, anionic, nonionic, cationic and amphoteric fixing and nonfixing polymers, mineral, vegetable and synthetic oils, ceramides, pseudoceramides, volatile and nonvolatile, linear and cyclic and modified and unmodified silicones, and any other additive conventionally used in cosmetic compositions intended in particular to be applied to the hair.

For example, use can be made, as an additive, of an anionic fixing polymer which may or not comprise silicone.

Of course, a person skilled in the art will take care to choose the optional compound or compounds to be added to the composition according to the invention so that at least one advantageous property intrinsically attached to the composition in accordance with the invention is not, or not substantially, detrimentally affected by the envisaged addition.

These compositions can be provided in various forms, as lotions, gels, milks, creams or mousses, and can, for example, be applied from pump-action sprays or in aerosol containers to provide for application of the composition in the vaporized form or in the mousse form. Such packaging forms are indicated, for example, when it is desired to obtain a spray, a lacquer or a mousse for fixing or treating the hair. The compositions in accordance with the invention can also be provided in the form of creams, gels, emulsions, lotions or waxes.

When the composition according to the invention is packaged in the form of an aerosol for the purpose of obtaining a lacquer or a mousse, it comprises at least one propellant which can be chosen from volatile hydrocarbons, such as n-butane, propane, isobutane or pentane, a chlorinated and/or fluorinated hydrocarbon, and their mixtures. Use may also be made, as propellant, of carbon dioxide gas, nitrous oxide, dimethyl ether (DME), nitrogen or compressed air. Use may also be made of mixtures of propellants. In one embodiment of the invention, the propellant is dimethyl ether.

The propellant can be, for example, present at a concentration ranging from 5 and 90% by weight with respect to the total weight of the composition in the aerosol device and, further, for example, at a concentration ranging from 10 to 60%.

The compositions of the invention can be used in hair products, lipsticks, nail varnishes or care creams.

The compositions in accordance with the invention can be applied to wet or dry hair.

The invention will be more fully illustrated with the help of the following nonlimiting examples.

All percentages are relative percentages by weight with respect to the total weight of the composition and a.m. means active material.

### EXAMPLE 1

Three compositions in accordance with the invention were prepared.

I) Preparation of the Film-forming Polymers

1) Preparation of the Film-forming Polymer P1

140 g of absolute ethanol were introduced into a 1 liter reactor equipped with a central stirrer, a reflux condenser and a nitrogen inlet and were brought to reflux (78° C.) for 45 minutes. The following materials were run into the reactor at reflux, simultaneously and over 2 hours:

60 g of absolute ethanol and 2.2 g of 2,5-bis(2-ethylhexanolperoxy)-2,5-dimethylhexane,

32 g of acrylic acid+68 g of methyl methacrylate+100 g of isobutyl acrylate.

At the end of the two additions, the reaction mixture was left at 78° C. for an additional 3 hours and was then allowed to return to ambient temperature with stirring. A viscous solution of polymer P1 at a concentration of 50% in absolute ethanol was obtained. The composition of the polymer P1 was, as percentage by weight with respect to the total weight of polymer:

| | |
|---|---|
| Acrylic acid | 16% |
| Methyl methacrylate | 34% |
| Isobutyl acrylate | 50% |

2) Preparation of the Film-forming Polymer P2

A synthetic route identical to the preceding one was used, except for the composition of the monomers which were run in, which was as follows: 32 g of acrylic acid+70 g of methyl methacrylate+29 g of methyl acrylate+20 g of isobutyl acrylate. A viscous solution of polymer P2 at a concentration of 50% in absolute ethanol was obtained. The composition of the polymer P2 was, as percentage by weight with respect to the total weight of polymer:

| | |
|---|---|
| Acrylic acid | 16% |
| Methyl methacrylate | 35% |
| Isobutyl acrylate | 20% |
| Methyl acrylate | 29% |

3) Preparation of the Film-forming, Polymer P3

The polymer P3 was prepared by carrying out the process in accordance with the invention for the manufacture of film-forming polymer. 140 g of absolute ethanol were introduced into a 1 liter reactor equipped with a central stirrer, a reflux condenser and a nitrogen inlet and were brought to reflux (78° C.) over 45 minutes. The following mixture: 32 g of acrylic acid+0.4 g of initiator+10 g of ethanol, was introduced at reflux over 45 minutes. The reaction medium was left at reflux for 15 minutes after this mixture had finished being run in. The following mixture: 70 g of methyl methacrylate+58 g of methyl acrylate+40 g of isobutyl acrylate+50 g of 510A+1.8 g of initiator, was subsequently introduced over 1 hour 30 minutes, still at reflux. The reaction medium was left to reflux for 3 hours after this mixture had finished being run in and was diluted in order to have a final concentration in the region of 20% in ethanol. The polymer P3 was thus obtained. The composition of the polymer P3 was, as percentage by weight with respect to the total weight of polymer:

| | |
|---|---|
| Acrylic acid | 16% |
| Methyl methacrylate | 35% |
| Isobutyl acrylate | 20% |
| Methyl acrylate | 29% |

II) Preparation of the Compositions

| Composition 1: | |
|---|---|
| Polymer P1 | 6% a.m. |
| 2-Amino-2-methyl-1-propanol | q.s. 100% neutralization |
| Ethanol | q.s. for 100 g |
| Composition 2: | |
| Polymer P2 | 6% a.m. |
| 2-Amino-2-methyl-1-propanol | q.s. 100% neutralization |
| Ethanol | q.s. for 100 g |
| Composition 3: | |
| Polymer P3 | 6% a.m. |
| 2-Amino-2-methyl-1-propanol | q.s. 100% neutralization |
| Ethanol | q.s. for 100 g |

III) Characterization of the Compositions

The parameters defining the mechanical profile of the compositions are collated in Table I below. The acid number also appears in this Table I.

TABLE 1

| | | | Instantaneous | Recovery at 300 |
|---|---|---|---|---|
| | Acid | Elongation ε | recovery R₁ | seconds, R₃₀₀ |
| Composition | number | (in %) | (in %) | (in %) |
| 1 | 124 | >1500 | 25 | 35 |
| 2 | 124 | 1200 | 35 | 55 |
| 3 | 124 | 1200 | 48 | 61 |

Compositions 1 to 3 in accordance with the invention were applied to locks of natural European chestnut-brown hair.

After packaging these compositions in pump-action sprays and applications with 10 squeezes to locks of natural hair weighing 5 grams washed and dried beforehand, it was found that these compositions indeed fixed the hairstyle, gave the hair a pleasant coated feel and were properly removed on shampooing.

What is claimed is:

1. A cosmetic composition comprising, in a cosmetically acceptable medium, at least one film-forming polymer (A) chosen so that a material, obtained by drying said at least one polymer (A) in an aqueous or alcoholic medium at ambient temperature and at a relative humidity of 50%±5%, exhibits a mechanical profile defined by at least:
(a) an elongation at break (ε_{b}) of greater than or equal to 160%;
(b) an instantaneous recovery (Rᵢ) of from 25 to 70% after an elongation of 150%; and
(c) a recovery (R₃₀₀) at 300 seconds of from 30 to 100% after an elongation of 150%;
wherein said at least one film-forming polymer (A) comprises at least one monomeric residue comprising an unsaturated acid unit, the acid number of which is greater than 82.

2. A composition according to claim 1, wherein said acid number ranges from 82 to 235.

3. A composition according to claim 2, wherein said acid number ranges from 90 to 190.

4. A composition according to claim 3, wherein said acid number ranges from 100 to 130.

5. A composition according to claim 1, wherein said at least one film-forming polymer (A) is present at a concentration ranging from 0.05 to 20% by weight with respect to the total weight of the composition.

6. A composition according to claim 5, wherein said at least one film-forming polymer (A) is present at a concentration ranging from 0.1 to 15% by weight with respect to the total weight of the composition.

7. A composition according to claim 6, wherein said at least one film-forming polymer (A) is present at a concentration ranging from 0.25 to 10% by weight with respect to the total weight of the composition.

8. A composition according to claim 1, wherein said at least one monomeric residue is chosen from monomeric residues comprising at least one unsaturated carboxylic acid and monomeric residues comprising at least one unsaturated sulphonic acid.

9. A composition according to claim 1, further comprising at least one cosmetic additive chosen from thickeners, anionic, nonionic, cationic and amphoteric surfactants, fragrances, preservatives, sunscreen agents, proteins, vitamins, provitamins, anionic, nonionic, cationic and amphoteric fixing and nonfixing polymers, mineral, vegetable and synthetic oils, ceramides, pseudoceramides, and volatile and nonvolatile, linear and cyclic and modified and unmodified silicones.

10. An aerosol device, comprising a cosmetic composition comprising, in a cosmetically acceptable medium, at least one film-forming polymer (A) chosen so that a material, obtained by drying said at least one polymer (A) in an aqueous or alcoholic medium at ambient temperature and at a relative humidity of 50%±5%, exhibits a mechanical profile defined by at least:
(a) an elongation at break (ε_{b}) of greater than or equal to 160%;
(b) an instantaneous recovery (Rᵢ) of from 25 to 70% after an elongation of 150%; and
(c) a recovery (R₃₀₀) at 300 seconds of from 30 to 100% after an elongation of 150%;
wherein said at least one film-forming polymer (A) comprises at least one monomeric residue comprising an unsaturated acid unit, the acid number of which is greater than 82.

11. A process for retaining the form of or shaping a hairstyle, comprising applying to the hair a cosmetic composition comprising, in a cosmetically acceptable medium, at least one film-forming polymer (A) chosen so that a material, obtained by drying said at least one polymer (A) in an aqueous or alcoholic medium at ambient temperature and at a relative humidity of 50%±5%, exhibits a mechanical profile defined by at least:
(a) an elongation at break (ε_{b}) of greater than or equal to 160%;
(b) an instantaneous recovery (Rᵢ) of from 25 to 70% after an elongation of 150%; and
(c) a recovery (R₃₀₀) at 300 seconds of from 30 to 100% after an elongation of 150%;
wherein said at least one film-forming polymer (A) comprises at least one monomeric residue comprising an unsaturated acid unit, the acid number of which is greater than 82,
and further wherein said cosmetic composition is applied to the hair in an amount effective to retain the form of or shape a hairstyle .

12. A process for the manufacture of at least one film-forming polymer, comprising:
first polymerizing at least one monomer (i) comprising at least one hydrophilic unit to obtain a polymer rich in hydrophilic units;
subsequently polymerizing the polymer rich in hydrophilic units with a mixture of at least one hard monomer (ii) and at least one soft monomer (iii) to obtain said at least one film-forming polymer;
said monomers (i), (ii) and (iii) being used in the following proportions, defined by weight with respect to the total weight of the monomeric residues in the at least one film-forming polymer:
10 to 50% of said at least one monomer (i) comprising at least one hydrophilic unit;
20 to 50% of said at least one hard monomer (ii); and
30 to 70% of said at least one soft monomer (iii),
wherein the first and subsequent polymerizations are carried out in an alcoholic or aqueous/alcoholic solvent medium in the presence of a free radical initiator at a temperature ranging from 60° C. to 90° C.

13. A process according to claim 12, wherein said at least one monomer (i) comprising at least one hydrophilic unit is chosen from acrylic acid, methacrylic acid and itaconic acid.

14. A process according to claim 13, wherein said at least one monomer (i) is acrylic acid.

15. A process according to claim 12, wherein said at least one hard monomer (ii) is methyl methacrylate.

16. A process according to claim 12, wherein said at least one soft monomer (iii) is chosen from methyl acrylate and isobutyl acrylate.

17. At least one film-forming polymer, obtained by a process comprising:
first polymerizing at least one monomer (i) comprising at least one hydrophilic unit to obtain a polymer rich in hydrophilic units;
subsequently polymerizing the polymer rich in hydrophilic units with a mixture of at least one hard monomer (ii) and of at least one soft monomer (iii) to obtain said at least one film-forming polymer;
said monomers (i), (ii) and (iii) being used in the following proportions, defined by weight with respect to the total weight of the monomeric residues in the at least one film-forming polymer:
10 to 50% of said at least one monomer (i) comprising at least one hydrophilic unit;
20 to 50% of said at least one hard monomer (ii); and
30 to 70% of said at least one soft monomer (iii),
wherein the first and subsequent polymerizations are carried out in an alcoholic or aqueous/alcoholic solvent medium in the presence of a free radical initiator at a temperature ranging from 60° C. to 90° C.

18. A cosmetic composition comprising, in a cosmetically acceptable medium, at least one film-forming polymer obtained by a process comprising:
first polymerizing at least one monomer (i) comprising at least one hydrophilic unit to obtain a polymer rich in hydrophilic units;
subsequently polymerizing the polymer rich in hydrophilic units with a mixture of at least one hard monomer (ii) and of at least one soft monomer (iii) to obtain said at least one film-forming polymer;
said monomers (i), (ii) and (iii) being used in the following proportions, defined by weight with respect to the total weight of the monomeric residues in the at least one film-forming polymer:
10 to 50% of said at least one monomer (i) comprising at least one hydrophilic unit;
20 to 50% of said at least one hard monomer (ii); and
30 to 70% of said at least one soft monomer (iii),
wherein the first and subsequent polymerizations are carried out in an alcoholic or aqueous/alcoholic solvent medium in the presence of a free radical initiator at a temperature ranging from 60° C. to 90° C.

19. A lipstick, a nail varnish or a care cream comprising, in a cosmetically acceptable medium, at least one film-forming polymer (A) chosen so that a material, obtained by drying said at least one polymer (A) in an aqueous or alcoholic medium at ambient temperature and at a relative humidity of 50%±5%, exhibits a mechanical profile defined by at least:
(a) an elongation at break (ε_{b}) of greater than or equal to 160%;
(b) an instantaneous recovery (Rᵢ) of from 25 to 70% after an elongation of 150%; and
(c) a recovery (R₃₀₀) at 300 seconds of from 30 to 100% after an elongation of 150%;
wherein said at least one film-forming polymer (A) comprises at least one monomeric residue comprising an unsaturated acid unit, the acid number of which is greater than 82.

20. A lipstick, a nail varnish or a care cream comprising, in a cosmetically acceptable medium, at least one film-forming polymer obtained by a process comprising:
first polymerizing at least one monomer (i) comprising at least one hydrophilic unit to obtain a polymer rich in hydrophilic units;
subsequently polymerizing the polymer rich in hydrophilic units with a mixture of at least one hard monomer (ii) and of at least one soft monomer (iii) to obtain said at least one film-forming polymer;
said monomers (i), (ii) and (iii) being used in the following proportions, defined by weight with respect to the total weight of the monomeric residues in the at least one film-forming polymer:
10 to 50% of said at least one monomer (i) comprising at least one hydrophilic unit;
20 to 50% of said at least one hard monomer (ii); and
30 to 70% of said at least one soft monomer (iii),
wherein the first and subsequent polymerizations are carried out in an alcoholic or aqueous/alcoholic solvent medium in the presence of a free radical initiator at a temperature ranging from 60° C. to 90° C.

21. A hair care composition comprising, in a cosmetically acceptable medium, at least one film-forming polymer (A) chosen so that a material, obtained by drying said at least one polymer (A) in an aqueous or alcoholic medium at ambient temperature and at a relative humidity of 50%±5%, exhibits a mechanical profile defined by at least:
(a) an elongation at break (ε_{b}) of greater than or equal to 160%;
(b) an instantaneous recovery (Rᵢ) of from 25 to 70% after an elongation of 150%; and
(c) a recovery (R₃₀₀) at 300 seconds of from 30 to 100% after an elongation of 150%;
wherein said at least one film-forming polymer (A) comprises at least one monomeric residue comprising an unsaturated acid unit, the acid number of which is greater than 82.

22. A hair care composition comprising, in a cosmetically acceptable medium, at least one film-forming polymer obtained by a process comprising:
first polymerizing at least one monomer (i) comprising at least one hydrophilic unit to obtain a polymer rich in hydrophilic units;
subsequently polymerizing the polymer rich in hydrophilic units with a mixture of at least one hard monomer (ii) and of at least one soft monomer (iii) to obtain said at least one film-forming polymer;
said monomers (i), (ii) and (iii) being used in the following proportions, defined by weight with respect to the total weight of the monomeric residues in the at least one film-forming polymer:
10 to 50% of said at least one monomer (i) comprising at least one hydrophilic unit;
20 to 50% of said at least one hard monomer (ii); and
30 to 70% of said at least one soft monomer (iii),
wherein the first and subsequent polymerizations are carried out in an alcoholic or aqueous/alcoholic solvent medium in the presence of a free radical initiator at a temperature ranging from 60° C. to 90° C.

23. A lacquer, a spray or a mousse comprising, in a cosmetically acceptable medium, at least one film-forming polymer (A) chosen so that a material, obtained by drying said at least one polymer (A) in an aqueous or alcoholic medium at ambient temperature and at a relative humidity of 50%±5%, exhibits a mechanical profile defined by at least:
(a) an elongation at break (ε_{b}) of greater than or equal to 160%;
(b) an instantaneous recovery (Rᵢ) of from 25 to 70% after an elongation of 150%; and
(c) a recovery (R₃₀₀) at 300 seconds of from 30 to 100% after an elongation of 150%;
wherein said at least one film-forming polymer (A) comprises at least one monomeric residue comprising an unsaturated acid unit, the acid number of which is greater than 82.

24. A lacquer, a spray or a mousse comprising, in a cosmetically acceptable medium, at least one film-forming obtained by a process comprising:
first polymerizing at least one monomer (i) comprising at least one hydrophilic unit to obtain a polymer rich in hydrophilic units;
subsequently polymerizing the polymer rich in hydrophilic units with a mixture of at least one hard monomer (ii) and of at least one soft monomer (iii) to obtain said at least one film-forming polymer;
said monomers (i), (ii) and (iii) being used in the following proportions, defined by weight with respect to the total weight of the monomeric residues in the at least one film-forming polymer:
10 to 50% of said at least one monomer (i) comprising at least one hydrophilic unit;
20 to 50% of said at least one hard monomer (ii); and
30 to 70% of said at least one soft monomer (iii),
wherein the first and subsequent polymerizations are carried out in an alcoholic or aqueous/alcoholic solvent medium in the presence of a free radical initiator at a temperature ranging from 60° C. to 90° C.

25. A cream, gel, lotion, emulsion or wax comprising, in a cosmetically acceptable medium, at least one film-forming polymer (A) chosen so that a material, obtained by drying said at least one polymer (A) in an aqueous or alcoholic medium at ambient temperature and at a relative humidity of 50%±5%, exhibits a mechanical profile defined by at least:
(a) an elongation at break (ε_{b}) of greater than or equal to 160%;
(b) an instantaneous recovery (Rᵢ) of from 25 to 70% after an elongation of 150%; and
(c) a recovery (R₃₀₀) at 300 seconds of from 30 to 100% after an elongation of 150%;
wherein said at least one film-forming polymer (A) comprises at least one monomeric residue comprising an unsaturated acid unit, the acid number of which is greater than 82.

26. A cream, gel, lotion, emulsion or wax comprising, in a cosmetically acceptable medium, at least one film-forming polymer obtained by a process comprising:
first polymerizing at least one monomer (i) comprising at least one hydrophilic unit to obtain a polymer rich in hydrophilic units;
subsequently polymerizing the polymer rich in hydrophilic units with a mixture of at least one hard monomer (ii) and of at least one soft monomer (iii) to obtain said at least one film-forming polymer;
said monomers (i), (ii) and (iii) being used in the following proportions, defined by weight with respect to the total weight of the monomeric residues in the at least one film-forming polymer:
10 to 50% of said at least one monomer (i) comprising at least one hydrophilic unit;
20 to 50% of said at least one hard monomer (ii); and
30 to 70% of said at least one soft monomer (iii),
wherein the first and subsequent polymerizations are carried out in an alcoholic or aqueous/alcoholic solvent medium in the presence of a free radical initiator at a temperature ranging from 60° C. to 90° C.
